SEQUENCE LISTING (4 sequences)

SEQ ID NO: 1 - DNA, 20 bp, Artificial Sequence
  forward primer for 5-HTTLPR
ggcgttgccgctctgaatgc

SEQ ID NO: 2 - DNA, 23 bp, Artificial Sequence
  reverse primer for 5-HTTLPR
gagggactgagctggacaaccac

SEQ ID NO: 3 - DNA, 21 bp, Artificial Sequence
  forward primer for STin2 VNTR
gtcagtatcacaggctgcgag

SEQ ID NO: 4 - DNA, 23 bp, Artificial Sequence
  reverse primer for STin2 VNTR
tgttcctagtcttacgccagtgg

(19) United States
(12) Patent Application Publication
(10) Pub. No.: US 2020/0377947 A1
(43) Pub. Date: Dec. 3, 2020

(54) METHOD FOR EXAMINING LONG-TERM PROGNOSIS OF ACUTE CORONARY SYNDROME AND DIAGNOSTIC KIT

(71) Applicant: INDUSTRY FOUNDATION OF CHONNAM NATIONAL UNIVERSITY, Gwangju (KR)

(72) Inventor: Jae Min KIM, Gwangju (KR)

(21) Appl. No.: 16/762,999

(22) PCT Filed: Aug. 23, 2018
(86) PCT No.: PCT/KR2018/009740

§ 371 Date: May 11, 2020

(30) Foreign Application Priority Data
May 16, 2018 (KR) 10-2018-0056108

(51) Int. Cl. C12Q 1/6883

(52) U.S. Cl. CPC C12Q 1/6883; C12Q 2600/118; C12Q 2600/156

(57) ABSTRACT

A diagnostic method according to an embodiment of the present invention is to determine prognosis, such as recurrence and death, which commonly occurs after acute coronary syndrome. A method of determining long-term prognosis of acute coronary syndrome includes analyzing anxiety and serotonin transporter gene polymorphism associated therewith. The method is capable of determining the risk of incidence of major adverse cardiac events including recurrence and/or death after acute coronary syndrome.

### TECHNICAL FIELD

The present invention relates to a diagnostic method for determining prognosis, such as recurrence and death, which commonly occurs after acute coronary syndrome, and more particularly to a method of determining long-term prognosis of acute coronary syndrome, which is capable of determining the risk of incidence of major adverse cardiac events including recurrence and/or death after acute coronary syndrome by analyzing anxiety and serotonin transporter gene polymorphism associated therewith, and a diagnostic kit.

### BACKGROUND ART

Although it is currently possible to prevent the recurrence of acute coronary syndrome to some extent through treatment with antithrombotic agents and cholesterol-reducing agents (statins), etc., major clinical trials have indicated that statins prevent recurrence in about 30% of patients but that treatment with the above kinds of drugs cannot prevent recurrence in the remaining 70% of patients. Hence, new treatment protocols that prevent the recurrence of acute coronary syndrome in all patients are needed, but currently no such treatment exists. Under these circumstances, the prevention of outbreaks through accurate identification and intensive management of high-risk cases, or the prevention of sudden out-of-hospital outbreaks is thought to be useful for improving life expectancy and prognosis.

The prognosis of recurrence of acute coronary syndrome is clinically determined using a mechanical test, such as a cardiac catheter test, including coronary angiography or left ventricular angiography, or using a biomarker. However, the former is not easy to implement in terms of time and cost, and consequently places a heavy burden on patients. In addition, these methods do not provide complete information in view of predicting recurrence.

For example, studies to date indicate that the risk of recurrence cannot be predicted based on the extent of stenosis of coronary arteries in coronary angiography (Levine G. N., et al.: N. Engl. J. Med., 1995, Vol. 332, pp. 512-521: Libby, Circulation, 1995, Vol. 91, pp. 2844-2850). Moreover, in other studies, acute stage markers such as troponin T, HFABP and high-sensitivity CRP, or risk-factor markers such as total cholesterol, triglyceride, HDL cholesterol and remnant lipoprotein (RPL) cholesterol, are used, but are not sufficiently effective as markers for predicting reoccurrence for reasons of sensitivity and specificity (M. Panteghini, Role and importance of biochemical markers in clinical cardiology: European Heart J., 2004, Vol.25, pp. 1187-1196).

### DISCLOSURE

### Technical Problem

The present inventors have performed research to determine the correlation between anxiety and major adverse cardiac events in acute coronary syndrome, thus culminating in the present invention.

Accordingly, an objective of the present invention is to provide a method of determining long-term prognosis of acute coronary syndrome, which may contribute to a decision-making process with regard to therapeutic drugs or treatment methods, by confirming that serotonin transporter (5-HTT) gene polymorphism may be used as a biomarker for predicting long-term prognosis of acute coronary syndrome, making it possible to predict the possibility of incidence of major adverse cardiac events including recurrence and/or death after acute coronary syndrome.

Another objective of the present invention is to provide a kit for diagnosing long-term prognosis of acute coronary syndrome, in which baseline anxiety in patients with acute coronary syndrome and serotonin transporter gene polymorphisms in biosamples are analyzed, thus predicting the possibility of incidence of major adverse cardiac events including recurrence and/or death after acute coronary syndrome in patients with acute coronary syndrome, thereby enabling the preemptive prevention of recurrence and/or death after acute coronary syndrome in patients, which is clinically useful.

The objectives of the present invention are not limited to the foregoing, and it is to be understood that other objectives not mentioned herein can be clearly anticipated by those skilled in the art from the following description.

### Technical Solution

In order to accomplish the above objectives, the present invention provides a method of determining long-term prognosis of acute coronary syndrome, including an investigation step of confirming whether a patient with acute coronary syndrome has anxiety at baseline, a genotyping step of confirming whether a biomarker for diagnosing long-term prognosis of acute coronary syndrome contained in a biological sample of the patient is present, and a decision step of determining the possibility of incidence of a major adverse cardiac event including recurrence or death after acute coronary syndrome based on whether the patient has anxiety and whether the biomarker for determining long-term prognosis of acute coronary syndrome is present.

In a preferred embodiment, the biomarker for determining long-term prognosis of acute coronary syndrome is a STin2 VNTR 9/12 or 12/12 genotype of VNTR (variable-number tandem repeat) polymorphism in the second intron site of a serotonin transporter (5-HTT) gene [STin2 VNTR gene polymorphism].

In a preferred embodiment, the decision step is performed in a manner in which the possibility of experiencing a major adverse cardiac event 5 years after baseline is determined to be higher in a patient in which at least one of STin2 VNTR 9/12 and 12/12 genotypes is analyzed to be present in the sample of the patient confirmed to have anxiety at baseline in the investigation step and confirmed to have anxiety in the genotyping step than in a patient in which the STin2 VNTR gene polymorphism is absent in the sample of the patient confirmed to have anxiety in the investigation step.

In a preferred embodiment, the possibility of experiencing a major adverse cardiac event 5 years after baseline in a patient confirmed to have anxiety but having a STin2 VNTR 10/12 genotype of the STin2 VNTR gene polymorphism is equivalent to that of a patient confirmed to have no anxiety.

In a preferred embodiment, the genotyping step includes isolating DNA containing the second intron site [STin2] of a serotonin transporter (5-HTT) gene isolated from the patient with anxiety, amplifying the isolated DNA using a sense primer and an antisense primer, and investigating the presence or absence of at least one of STin2 VNTR 9/12, STin2 VNTR 10/12, and STin2 VNTR 12/12 genotypes by examining the amplified DNA using a restriction enzyme that recognizes mutation in the STin2 VNTR (variable-number tandem repeat).

In a preferred embodiment, the biological sample is selected from among a tissue and a body fluid including blood.

In addition, the present invention provides a diagnostic kit for determining long-term prognosis of acute coronary syndrome, including a genotyping unit for analyzing STin2 VNTR gene polymorphism of a patient with acute coronary syndrome investigated to have high anxiety at baseline.

In a preferred embodiment, the genotyping unit uses a polymerase chain reaction (PCR).

In a preferred embodiment, the possibility of experiencing a major adverse cardiac event is higher in a patient confirmed to have a STin2 VNTR 9 or 12/12 genotype than in a patient confirmed to have a STin2 VNTR 10/12 genotype using the genotyping unit.

In a preferred embodiment, the diagnostic kit is a microarray.

### Advantageous Effects

According to the present invention, it is confirmed that serotonin transporter (5-HTT) gene polymorphism can be used as a biomarker for predicting long-term prognosis of acute coronary syndrome, thereby providing a biomarker that can relatively accurately determine long-term prognosis of acute coronary syndrome 5 years after baseline.

In addition, the method of determining long-term prognosis of acute coronary syndrome according to the present invention enables the prediction and/or diagnosis of the possibility of onset of major adverse cardiac events 5 years after baseline and can thus contribute to the decision-making process with regard to therapeutic drugs or treatment methods.

Moreover, among samples of patients with acute coronary syndrome confirmed to have anxiety at baseline according to the present invention, serotonin transporter gene polymorphism is analyzed, thus predicting the possibility of incidence of major adverse cardiac events including recurrence and/or death after acute coronary syndrome in patients with acute coronary syndrome, thereby enabling the preemptive prevention of recurrence and/or death after acute coronary syndrome in patients, which is clinically useful.

The effects of the present invention are not limited to the foregoing, and it is to be understood that other objectives not mentioned herein can be clearly anticipated by those skilled in the art from the following description.

### BRIEF DESCRIPTION OF THE DRAWING

FIG. 1 shows the effects of anxiety and serotonin transporter gene polymorphism on major adverse cardiac events during the follow-up of 5 to 12 years of patients with acute coronary syndrome.

### MODE FOR INVENTION

The terminology used in the present invention is merely used to describe particular embodiments, and is not intended to limit the present invention. As used herein, the singular forms are intended to include the plural forms as well, unless the context clearly indicates otherwise. It will be understood that the terms “comprise”, “include”, “have”, etc. when used in this specification specify the presence of stated features, integers, steps, operations, elements, components, or combinations thereof, but do not preclude the presence or addition of one or more other features, integers, steps, operations, elements, components, or combinations thereof.

It will be further understood that, although terms such as “first”, “second”, etc. may be used herein to describe various elements, these elements are not to be limited by these terms. These terms are only used to distinguish one element from another element. For instance, a “first” element discussed below could be termed a “second” element without departing from the scope of the present invention. Similarly, the “second” element could also be termed a “first” element.

Unless otherwise defined, all terms including technical and scientific terms used herein have the same meanings as those commonly understood by one of ordinary skill in the art to which the present invention belongs. It will be further understood that the terms used herein should be interpreted as having meanings consistent with their meanings in the context of this specification and the relevant art, and are not to be interpreted in an idealized or overly formal sense unless expressly so defined herein.

In interpreting elements, it is to be understood that an error range is included even if there is no separate description thereof.

In the case of a description of a temporal relationship, for example, when the temporal relationship is described as ‘after’, ‘following’, ‘subsequently’, ‘before’, etc., this includes non-consecutive cases, unless ‘immediately’ or ‘directly’ is used.

As used herein, the term “diagnosis” means identifying the presence or characteristic of a pathological condition. With regard to the purpose of the present invention, “diagnosis” means determining the long-term prognosis of acute coronary syndrome based on in-vitro analysis of body fluids, that is, the possibility of incidence of major adverse cardiac events five years after baseline.

As used herein, the term “biomarker” means a substance that may indicate a disease state. In the context of the present invention regarding the diagnosis of long-term prognosis of acute coronary syndrome, the “biomarker” is at least one of STin2 VNTR 9/12 and STin2 VNTR 12/12 genotypes of the VNTR (variable-number tandem repeat) polymorphism in the second intron site of a serotonin transporter (5-HTT) gene [STN2 VNTR gene polymorphism]. Among patients with acute coronary syndrome confirmed to have anxiety at baseline, patients having a “biomarker” are more likely to experience major adverse cardiac events 5 years after baseline.

As used herein, the term “blood” includes whole blood, serum and plasma.

As used herein, the term “biological sample” includes various types of samples obtained from an individual, and may also be used in diagnosis or monitoring analysis. Biological fluid samples include blood, cerebrospinal fluid (CSF), urine, and other liquid samples of biological origin. For example, the sample may be pretreated for concentration and separation, if necessary.

As used herein, the term “individual” is a mammal, preferably a human, and the terms “individual” and “subject” may be used interchangeably in the present invention.

As used herein, the term “baseline” refers to the time point at which initial medical treatment is performed after the onset of acute coronary syndrome, and preferably a time point within 2 weeks (average 7 days) of inpatient treatment after the onset of acute coronary syndrome.

Hereinafter, a detailed description will be given of the technical configuration of the present invention with reference to the accompanying drawings and preferred embodiments.

However, the present invention is not limited to the embodiments described herein, and may be embodied in other forms. Throughout the specification, the same reference numerals used to explain the present invention designate the same elements.

The present inventors have ascertained that serotonin transporter (5-HTT) gene polymorphism may be used as a biomarker for predicting long-term prognosis of acute coronary syndrome, and thus the present invention provides a method of determining long-term prognosis of acute coronary syndrome using, as a biomarker, the gene polymorphism in the second intron site of the serotonin transporter gene in order to predict and/or diagnose the possibility of incidence of major adverse cardiac events including recurrence and/or death after acute coronary syndrome, and a diagnostic kit.

Specifically, anxiety is common in acute coronary syndrome, but the effect of anxiety on long-term cardiac prognosis is not as obvious as that of genetic vulnerability Because the association between anxiety in patients with acute coronary syndrome and major adverse cardiac event after years has been controversial. Moreover, the serotonin transporter (5-HTT) gene acts on the central nervous system and the cardiovascular system, potentially affecting both anxiety and acute coronary syndrome, but the specific effect thereof on the long-term prognosis of acute coronary syndrome is unknown.

As noted above, the 5-HTT gene is called SLC6A4 and is located on chromosome 17q12. To date, this 5-HTT gene has been reported to have polymorphisms at two sites. The first is a 44-bp insertion/deletion polymorphism in the promoter region (5-HTT gene linked polymorphic region; 5-HTTLPR), and the second is a VNTR (variable-number tandem repeat; Stin2.9, Stin2.10, Stin2.12) polymorphism in the second intron site.

In the present invention, as described below, the effect of VNTR (variable-number tandem repeat; Stin2.9, Stin2.10, Stin2.12) polymorphism in the second intron site, which is a second polymorphism of the serotonin transporter (5-HTT) gene, on the correlation between anxiety and major adverse cardiac events in acute coronary syndrome is clearly confirmed.

Therefore, the method of determining long-term prognosis of acute coronary syndrome according to the present invention includes an investigation step of confirming whether a patient with acute coronary syndrome has anxiety at baseline, a genotyping step of confirming whether a biomarker for diagnosing long-term prognosis of acute coronary syndrome contained in the biological sample of the patient is present, and a decision step of determining the possibility of incidence of major adverse cardiac events including recurrence or death after acute coronary syndrome based on whether the patient has anxiety and whether the biomarker for determining long-term prognosis of acute coronary syndrome is present.

More specifically, the biomarker for determining long-term prognosis of acute coronary syndrome analyzed in the genotyping step is the VNTR (variable-number tandem repeat) polymorphism in the second intron site of the serotonin transporter (5-HTT) gene [STin2 VNTR gene polymorphism], and is particularly a STin2 VNTR 9/12 or 12/12 genotype. Here, the presence of at least one of STin2 VNTR 9/12 and 12/12 genotypes may indicate the risk of onset of major adverse cardiac events 5 years after baseline.

Furthermore, the genotyping step includes isolating DNA containing the second intron site [STin2] of a serotonin transporter (5-HTT) gene isolated from a patient with anxiety, amplifying the isolated DNA using a sense primer and an antisense primer, and investigating the presence or absence of at least one of STin2 VNTR 9/12, STin2 VNTR 10/12, and STin2 VNTR 12/12 genotypes by examining the amplified DNA using a restriction enzyme that may recognize mutation in the STin2 VNTR (variable-number tandem repeat). Here, the analysis method used in the genotyping step may include other known methods useful for identifying the presence of a biomarker for determining long-term prognosis of acute coronary syndrome. In the present invention, the diagnostic method may be performed both in vitro and/or in vivo, but preferably the diagnostic method of the present invention is an in-vitro method based on a sample obtained from an individual and provided in vitro. As such, the biological sample may be selected from among a tissue and a body fluid including blood.

Also, the decision step is performed in a manner in which the possibility of experiencing major adverse cardiac events 5 years after baseline is determined to be higher in a patient in which at least one of STin2 VNTR 9/12 and 12/12 genotypes is analyzed to be present in the sample of the patient confirmed to have anxiety at baseline in the investigation step and confirmed to have anxiety in the measurement step than in a patient in which STin2 VNTR gene polymorphism is absent in the sample of the patient confirmed to have anxiety in the investigation step. Therefore, the presence of the biomarker for determining long-term prognosis of acute coronary syndrome in the biological sample of the patient with acute coronary syndrome confirmed to have anxiety at baseline may indicate the risk of onset of major adverse cardiac events 5 years after baseline.

Meanwhile, the possibility of experiencing major adverse cardiac events 5 years after baseline in a patient confirmed to have anxiety at baseline but having a STin2 VNTR 10/12 genotype of the STin2 VNTR gene polymorphism is equivalent to or less than that of a patient confirmed to have no anxiety. The effect of the STin2 VNTR 10/12 genotype has made clear the uncertain influence of anxiety on long-term cardiac prognosis in previous studies.

In addition, the present invention pertains to a diagnostic kit for determining long-term prognosis of acute coronary syndrome, which is used to determine the risk of onset of major adverse cardiac events 5 years after baseline in a patient with acute coronary syndrome confirmed to have anxiety at baseline. The diagnostic kit of the present invention includes a genotyping unit for analyzing the STin2 VNTR gene polymorphism of a biological sample, selected from among a tissue and a body fluid including blood, of a patient with acute coronary syndrome investigated to have anxiety at baseline. Here, the genotyping unit is able to use a polymerase chain reaction (PCR) or to use a monoclonal antibody against 5-methylcytosine. Moreover, the possibility of experiencing major adverse cardiac events may be determined to be higher in a patient confirmed to have a STin2 VNTR 9 or 12/12 genotype than in a patient confirmed to have a STin2 VNTR 10/12 genotype using the genotyping unit. Also, the diagnostic kit may be implemented in a microarray.

### EXAMPLE

1. Research subject

The baseline evaluation was performed on 969 inpatients who received revascularization within two weeks after acute coronary syndrome, recruited from a tertiary university hospital in Korea in 2007-2012. Details can be found on the website below.

(https://clinicaltrials.gov/ct2/show/NCT00419471)

2. Evaluation and statistical analysis

Anxiety was investigated using HADS-A, and scores higher than 7 were classified according to case level. Two 5-HTT genotypes were detected using polymerase chain reaction as follows.

(1) Analysis of deletion/insertion polymorphism in promoter region of serotonin transporter gene (5-HTTLPR)

For analysis of the deletion/insertion polymorphism in the promoter region of the serotonin transporter gene (5-HTTLPR), the primers used for polymerase chain reaction are shown in Table 1 below. PCR amplification was performed using 0.1 mM dNTP, 0.15 μM sense and antisense primers, 150 ng of genome DNA, 2 mM Tris⋅HCl (pH 7.5 at 25° C.), 10 mM KCl, 0.1 mM dithiothreitol (DTT), 0.01 mM EDTA, 0.05% Tween20 (trade name) (v/v), 0.05% Nonidet P40 (v/v), 5% glycerol, and 1.3μ expand high-fidelity PCR system enzyme mix (Boehringer Mannheim, Mannheim, Germany) under the following conditions. Denaturation at 95° C. for 4 min and then at 95° C. for 30 sec, annealing at 65° C. for 30 sec, and elongation at 72° C. for 45 sec were performed 10 times, after which denaturation at 95° C. for 30 sec, annealing at 65° C. for 30 sec, and elongation at 72° C. for 4 min 5 sec were performed 20 times. Elongation at 72° C. for 7 min was further conducted, and then the temperature was maintained at 4° C. The amplified products were identified with bands having 14 copies (s allele) and 16, 18, 20, 22 copies (defined as 1 allele when 16 copies or more) on a 2% agarose gel using a 100-bp ladder as a marker.

(2) Analysis of VNTR in second intron (intron2) of serotonin transporter gene

For VNTR analysis in intron 2 of the serotonin transporter gene, the VNTR site located in intron 2 of the serotonin transporter gene comprising 17 repeat sequences was amplified using polymerase chain reaction. The primers shown in Table 1 were used, and 20 ng of genome DNA, 50 mM KCl, 10 mM Tris⋅HCl (pH 9.0 at 25° C.), 0.1% Triton-X100 (trade name), 1 mM MgCl₂, 0.2 mM dNTP, 1μ Taq polymerase, and 1 μM of each of sense and antisense primers were mixed and allowed to react. For the PCR conditions, denaturation at 94° C. for 3 min and then at 94° C. for 30 sec, annealing at 60° C. for 45 sec, and elongation at 72° C. for 45 sec were repeated 25 times, after which elongation at 72° C. for 8 min was additionally conducted and then the temperature was maintained at 4° C. The amplified products were identified with bands having 9/12 and 10/12 copies (s alleles), and 12/12 copies (1 alleles), compared to a pUC 18 Hae III digestive marker (Sigma) on a 3% agarose gel.

TABLE 1

| | Forward (F) | |
|---|---|---|
| Poly- | and Reverse | PCR product |
| morphism | (R) primer | and allele |
| 5-HTTLPR | F:5′-GGCGTTGCC | 44bp 16, 18, 20 and 22 |
| | GCTCTGAATGC-3′ | copies of a repetitive |
| | R: 5′-GAGGGACT | sequence; l |
| | GAGCTGGACAACCA | 44bp 14 copy of a |
| | C-3′ | repetitive sequence: s |
| STin2 | F:5′-GTCAGTATC | 17bp 9 copy of a |
| VNTR | ACAGGCTGCGAG-3′ | repetitive sequence: 9 |
| | R:5′-TGTTCCTAG | 17bp 10 copy of a |
| | TCTTACGCCAGTG | repetitive sequence: 10 |
| | G-3′ | 17bp 12 copy of a |
| | | repetitive sequence: 12 |

5-HTTLPR: serotonin transporter gene linked promoter region;
STin2 VNTR: serotonin transporter intron 2 variable number tandem repeat

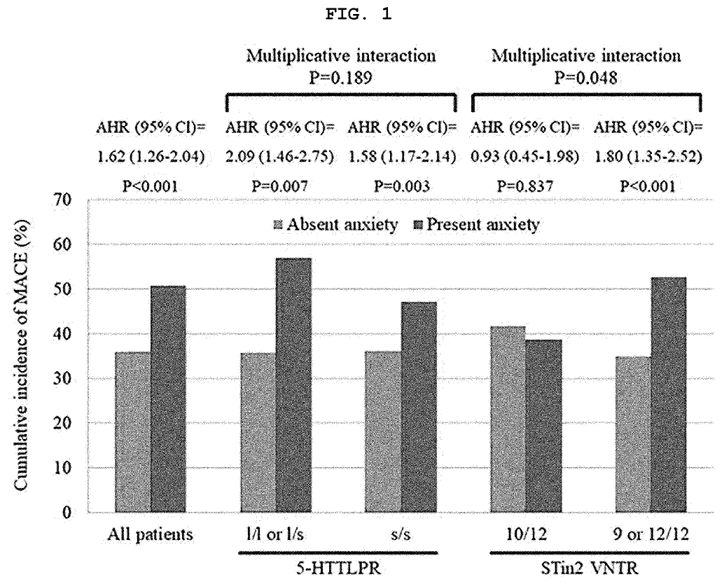

For 5-HTTLPR polymorphism, 1/1, 1/s and s/s were used, and for STin2 VNTR, 9/12, 10/12 and 12/12 were used. Also, statistical power was increased through subdivision into ‘1/1 or l/s’ vs ‘s/s’ and ‘10/12’ vs ‘9 or 12/12’. Baseline characteristics that potentially affect cardiac prognosis were evaluated. All patients were tracked for 5 to 12 years (average 8.7 years) until 2017 or death. The major adverse cardiac event, which is a primary outcome factor, was defined as all-cause mortality, myocardial infarction, or percutaneous coronary intervention. The Cox proportional hazards model was used after adjustment for potential covariates. The evaluation results thereof are shown in Table 2 below and in FIG. 1. Table 2 below shows the presence or absence of baseline anxiety, the number of major adverse cardiac events depending on the 5-HTT genotype, and the incidence rate thereof per patient-year (incidences/100 patient-years), after adjustment for covariates with a significant (p<0.05) correlation with anxiety or a major adverse cardiac event.

3. Results

As is apparent from Table 2 below, anxiety of a disability level was confirmed in 228 (23.5%) of 969 patients.

TABLE 2

| | | | | Number | Incidence | | | | |
|---|---|---|---|---|---|---|---|---|---|
| Patient | | | Number | Patient- | Rate per 100 | Crude HR | | | P Value for |
| Group | Anxiety | N | of Events | Years | Patient-Years | (95% Cl) | AHR (95% Cl) | P Value | Interaction |
| All patients | Absent | 741 | 267 | 6,447 | 4.1 | Reference | Reference | | |
| | Present | 228 | 116 | 1,983 | 5.8 | 1.83 (1.51-2.23) | 1.62 (1.26-2.04) | <0.001 | |
| 5-HTT-LPR | Absent | 315 | 113 | 2,742 | 4.1 | Reference | Reference | | 0.189 |
| II or I/s | Present | 86 | 49 | 747 | 6.6 | 2.09(1.46-2.75) | 1.66(1.15-2.40) | 0.007 | |
| s/s | Absent | 426 | 154 | 3,705 | 4.2 | Reference | Reference | | |
| | Present | 142 | 67 | 1,233 | 5.4 | 1.67 (1.21-2.27) | 1.58 (1.17-2.14) | 0.003 | |
| STin2VNTR | Absent | 127 | 53 | 1,104 | 4.8 | Reference | Reference | | 0.048 |
| 10/12 | Present | 31 | 12 | 270 | 4.4 | 1.11 (0.63-2.10) | 0.93 (0.45- 1.98) | 0.837 | |
| 9 or 12/12 | Absent | 614 | 214 | 5,346 | 4.0 | Reference | Reference | | |
| | Present | 197 | 104 | 1,710 | 6.1 | 1.98(1.62-2.73) | 1.80(1.35-2.52) | <0.001 | |

AHRs (95% Cls) were estimated adjusted for baseline age, sex, education, marital status, living alone, accommodation, employment,
Beck Depression Inventory Score, family history of depression, antidepressants taken, physical activity, sleep quality, hypertension, diabetes, smoking, ACS history, ACS diagnosis,
Killip class, left ventricular ejection fraction, Serum Troponin I, and serum creatine kinase-MB.
5HTT-LPR = serotonin transporter-linked promoter region; ACS = acute coronary syndrome; AHR = adjusted hazard ratio; Cl = confidence interval;
HR = hazard ratio; l = long; s = short; VNTR = variable number tandem repeat.

5-HTTLPR 1/1 or 1/s, and s/s genotypes were confirmed in 401 patients (41.4%) and 568 patients, respectively, and STin2 VNTR 10/12 and 9 or 12/12 genotypes were confirmed in 158 patients (16.3%) and 811 patients, respectively. The major adverse cardiac event was generated in 383 patients (39.5%).

As shown in FIG. 1, anxiety at baseline was associated with a higher risk of incidence of a major adverse cardiac event. There was a between-group difference in long-term major adverse cardiac events depending on the presence or absence of anxiety in patients having 9 or 12/12 gene polymorphism, and there was no group difference in patients having 10/12 polymorphism, indicating that the association between the baseline anxiety and the major adverse cardiac event is modified by STin2 VNTR. On the other hand, the incidence of major adverse cardiac events due to anxiety and 5-HTTLPR was not found.

However, the STin2 VNTR 9 or 12/12 genotype was associated with an increased risk of occurrence of anxiety. Although not shown in the examples, the effect of the 9 or 12/12 genotype on the risk/prognosis of acute coronary syndrome can lead to a reasonable conclusion that the 9 or 12/12 genotype raises 5-HTT transcription over 10/12 and is thus associated with poor cardiovascular prognosis.

Thus, it is deemed that the STin2 VNTR gene polymorphism negatively affects both anxiety and the cardiovascular system and thus can act as a regulator of anxiety after acute coronary syndrome and major adverse cardiac events.

The 5-HTTLPR gene polymorphism did not show such a regulatory effect. As a possible explanation therefor, the s allele of 5-HTTLPR is associated with anxiety sensitivity.

Specifically, the 1 allele causes an increase in platelet activation and thromboglobulin, which is associated with poor prognosis after acute coronary syndrome. Therefore, the effect of the s allele of 5-HTTLPR on anxiety vulnerability may be offset by a relatively beneficial effect on cardiac prognosis.

The above results show that the pathological impact of anxiety on poor cardiac prognosis depends on genetic vulnerability, which also means that the role of anxiety and the role of genetic predisposition are supported by the observed interactions.

Thus, the present invention may particularly provide the use of the STin2 VNTR gene polymorphism as a biomarker for determining long-term prognosis of acute coronary syndrome. Specifically, by evaluating the STin2 VNTR gene polymorphism at baseline, which is the acute stage of acute coronary syndrome, it is possible to improve the clinical prediction of long-term cardiac prognosis 5 years after baseline.

As described above, the use of the STin2 VNTR gene polymorphism as a biomarker for determining long-term prognosis of acute coronary syndrome in terms of clinical implications provides advantages of non-invasiveness and convenience, and thus the present invention can more easily identify the possibility of incidence of major adverse cardiac events, including recurrence and/or death after acute coronary syndrome.

Consequently, the present invention can not only contribute to a decision-making process with regard to therapeutic drugs or treatment methods, but also can be very helpful as a potential tool for preemptively preventing recurrence and/or death after acute coronary syndrome in a patient.

Although preferred embodiments of the present invention have been disclosed for illustrative purposes, those skilled in the art will appreciate that various modifications and substitutions are possible without departing from the scope and spirit of the invention as disclosed in the accompanying claims.

1. A method of determining long-term prognosis of acute coronary syndrome, comprising:
an investigation step of confirming whether a patient with acute coronary syndrome has anxiety at a baseline;
a genotyping step of confirming whether a biomarker for diagnosing long-term prognosis of acute coronary syndrome contained in a biological sample of the patient is present; and
a decision step of determining a possibility of incidence of a major adverse cardiac event including recurrence or death after acute coronary syndrome based on whether the patient has anxiety and whether the biomarker for determining long-term prognosis of acute coronary syndrome is present.

2. The method of claim 1, wherein the biomarker for determining long-term prognosis of acute coronary syndrome is a STin2 VNTR 9/12 or 12/12 genotype of VNTR (variable-number tandem repeat) polymorphism in a second intron site of a serotonin transporter (5-HTT) gene [STin2 VNTR gene polymorphism].

3. The method of claim 2, wherein the decision step is performed in a manner in which the possibility of experiencing the major adverse cardiac event 5 years after the baseline is determined to be higher in a patient in which at least one of STin2 VNTR 9/12 and 12/12 genotypes is analyzed to be present in a sample of the patient confirmed to have anxiety at the baseline in the investigation step and confirmed to have anxiety in the genotyping step than in a patient in which the STin2 VNTR gene polymorphism is absent in the sample of the patient confirmed to have anxiety in the investigation step.

4. The method of claim 3, wherein the possibility of experiencing the major adverse cardiac event 5 years after the baseline in a patient confirmed to have anxiety but having a STin2 VNTR 10/12 genotype of the STin2 VNTR gene polymorphism is equivalent to that of a patient confirmed to have no anxiety.

5. The method of claim 1, wherein the genotyping step comprises isolating DNA containing a second intron site [STin2] of a serotonin transporter (5-HTT) gene isolated from the patient with anxiety; amplifying the isolated DNA using a sense primer and an antisense primer; and investigating presence or absence of at least one of STin2 VNTR 9/12, STin2 VNTR 10/12, and STin2 VNTR 12/12 genotypes by examining the amplified DNA using a restriction enzyme that recognizes mutation in the STin2 VNTR (variable-number tandem repeat).

6. The method of claim 5, wherein the biological sample is selected from among a tissue and a body fluid including blood.

7. A diagnostic kit for determining long-term prognosis of acute coronary syndrome, comprising:
a genotyping unit for analyzing STin2 VNTR gene polymorphism of a patient with acute coronary syndrome investigated to have anxiety at a baseline.

8. The diagnostic kit of claim 7, wherein the genotyping unit uses a polymerase chain reaction (PCR).

9. The diagnostic kit of claim 7, wherein a possibility of experiencing a major adverse cardiac event is higher in a patient confirmed to have a STin2 VNTR 9 or 12/12 genotype than in a patient confirmed to have a STin2 VNTR 10/12 genotype using the genotyping unit.

10. The diagnostic kit of claim 7, wherein the diagnostic kit is a microarray.

11. The method of claim 2, wherein the genotyping step comprises isolating DNA containing a second intron site [STin2] of a serotonin transporter (5-HTT) gene isolated from the patient with anxiety; amplifying the isolated DNA using a sense primer and an antisense primer; and investigating presence or absence of at least one of STin2 VNTR 9/12, STin2 VNTR 10/12, and STin2 VNTR 12/12 genotypes by examining the amplified DNA using a restriction enzyme that recognizes mutation in the STin2 VNTR (variable-number tandem repeat).

12. The method of claim 3, wherein the genotyping step comprises isolating DNA containing a second intron site [STin2] of a serotonin transporter (5-HTT) gene isolated from the patient with anxiety; amplifying the isolated DNA using a sense primer and an antisense primer; and investigating presence or absence of at least one of STin2 VNTR 9/12, STin2 VNTR 10/12, and STin2 VNTR 12/12 genotypes by examining the amplified DNA using a restriction enzyme that recognizes mutation in the STin2 VNTR (variable-number tandem repeat).

13. The method of claim 4, wherein the genotyping step comprises isolating DNA containing a second intron site [STin2] of a serotonin transporter (5-HTT) gene isolated from the patient with anxiety; amplifying the isolated DNA using a sense primer and an antisense primer; and investigating presence or absence of at least one of STin2 VNTR 9/12, STin2 VNTR 10/12, and STin2 VNTR 12/12 genotypes by examining the amplified DNA using a restriction enzyme that recognizes mutation in the STin2 VNTR (variable-number tandem repeat).

14. The method of claim 11, wherein the biological sample is selected from among a tissue and a body fluid including blood.

15. The method of claim 12, wherein the biological sample is selected from among a tissue and a body fluid including blood.

16. 12. The method of claim 13, wherein the biological sample is selected from among a tissue and a body fluid including blood.

17. The diagnostic kit of claim 8, wherein the diagnostic kit is a microarray.

18. The diagnostic kit of claim 9, wherein the diagnostic kit is a microarray.
